(19) United States
(12) Patent Application Publication
(10) Pub. No.: US 2026/0216359 A1
(43) Pub. Date: Jul. 30, 2026

(54) CYCLIC PEPTIDE-BASED LYSOSOME TARGETING DEGRADERS

(71) Applicant: Wisconsin Alumni Research Foundation, Madison, WI (US)

(72) Inventors: Weiping Tang, Middleton, WI (US); Yaxian Zhou, Madison, WI (US); Yuan Zhao, Madison, WI (US)

(73) Assignee: Wisconsin Alumni Research Foundation, Madison, WI (US)

(21) Appl. No.: 19/147,631

(22) PCT Filed: Jan. 30, 2024
(86) PCT No.: PCT/US2024/013455

§ 371 Date: Jul. 11, 2025

Related U.S. Application Data
(60) Provisional application No. 63/482,445, filed on Jan. 31, 2023.

(51) Int. Cl. A61K 47/68; A61K 38/00; C07K 7/06

(52) U.S. Cl. CPC A61K 47/6811; A61K 47/6849; C07K 7/06; A61K 38/00

(57) ABSTRACT

Provided herein are bifunctional lysosomal targeting degraders that comprise a peptide ligand that binds to RGD-binding integrin as a shuttle molecule for lysosome degradation, and a protein binder that binds a membrane or extracellular protein of interest. The bifunctional degraders find use, e.g., for selectively targeted degradation of membrane and extracellular proteins via the endosomal/lysosomal pathway. Also provided herein are compositions comprising the bifunctional degraders, as well as methods of using the bifunctional degraders.

### CROSS-REFERENCE TO RELATED APPLICATIONS

Priority is hereby claimed to U.S. Provisional Application 63/482,445, filed Jan. 31, 2023, which is incorporated herein by reference in its entirety.

### FEDERAL FUNDING STATEMENT

This invention was made with government support under GM120357 and GM148266 awarded by the National Institutes of Health. The government has certain rights in the invention.

### SEQUENCE LISTING

The instant application contains a Sequence Listing which has been submitted in XML format and is hereby incorporated by reference in its entirety. The XML copy, created on Jan. 25, 2024 is named PCT-09824495-P220339WO01-APP-SEQ_LIST and is 14,322 bytes in size.

### BACKGROUND

Targeted Protein Degradation (TPD) is emerging as an exciting therapeutic option to confront diseases involving aberrantly expressed or mutated disease-causing proteins by engaging our body's natural protein disposal systems. TPD by chimeric molecules is a novel therapeutic modality (Deshaies, 2020, Nature. 580: 329-338). These chimeras are heterobifunctional molecules with one end binding to the protein of interest (POI) and the other end directing the resulting complex towards a certain degradation pathway. PROteolysis TArgeting Chimera (PROTAC) has received the most attention to date. (See Sakamoto et al., 2001, Proc. Natl. Acad. Sci. 98: 8554-8559; Luh et al., 2020, Angew. Chem. Int. Ed. 59: 15448-15466; Wu et al., 2020, Nat. Struct. Mol. Biol. 27: 605-614.) PROTACs contain an E3 ligase ligand to route the targeted protein to the proteasome for degradation. (Lai and Crews, 2017, Nat. Rev. Drug Discovery. 16: 101-114; Salami and Crews, 2017, Science. 355: 1163-1167; Cromm and Crews, 2017, Cell Chem. Biol. 24: 1181-1190; Toure and Crews, 2016, Angew. Chem. Int. Ed. 55: 1966-1973.) However, PROTACs are only capable of depleting intracellular proteins. There are many disease targets that are membrane or extracellular proteins.

To broaden the scope of targets, researchers have reported a way of tagging extracellular protein targets with a ligand for membrane receptors involved in active transport of molecules into the cell. The tagged protein is naturally shuttled to the lysosome in the cell where it is degraded. The bifunctional lysosome targeting degraders are generally created by conjugating ligands of the lysosome targeting receptors (LTRs) on the cell surface with ligands that can bind to the extracellular protein target. The LTRs employed in the past studies are carbohydrate binding proteins including cation-independent mannose 6-phosphate receptor (CIM6PR or insulin-like growth factor-II receptor) (Banik et al., 2020, Nature. 584: 291-297) and asialoglycoprotein receptor (ASGPR) (Zhou et al., 2021, ACS Cent. Sci. 7: 499-506; Ahn et al., 2021, Nat. Chem. Biol. 17: 937-946; Caianiello et al., 2021, Nat. Chem. Biol. 17: 947-953). The receptor-ligand interaction triggers the internalization of the extracellular proteins through receptor-mediated endocytosis, further inducing the degradation of the targets in the lysosome.

One type of bifunctional lysosome targeting degraders was developed by conjugating the ligand of the CIM6PR on the cell surface with a molecule that binds to the extracellular protein target (Banik et al., 2020, Nature. 584: 291-297). This type of bifunctional lysosome-targeting degrader that recruits CIM6PR was also termed LYsosome TArgeting Chimeras (LYTACs). CIM6PR is expressed ubiquitously in most cell types. The receptor-ligand interaction triggers the internalization of the extracellular proteins through receptor-mediated endocytosis, further inducing the degradation of the targets in the lysosome. CIM6PR is a transmembrane receptor that transports proteins bearing N-glycans capped with mannose 6-phosphate (M6P) residues to lysosomes (Ghosh et al., 2003, Nat. Rev. Mol. Cell Biol. 4: 202-213; Coutinho et al., 2012, Mol. Genet. Metab. 105: 542-550). Early studies showed that albumin modified with M6P increased the cellular uptake (Beljaars et al., 1999, Hepatology. 29: 1486-1493). Subsequently, CIM6PR was used to deliver therapeutic drugs conjugated with M6P derivatives for lysosomal enzyme replacement therapy and cancer treatment (Ghosh et al., 2003, Nat. Rev. Mol. Cell Biol. 4: 202-213; Gary-Bobo et al., 2007, Curr. Med. Chem. 14: 2945-2953). Various molecules, such as peptides, proteins, or liposomes, were covalently linked to the M6P or its analogues to achieve targeted drug delivery (Hoogendoorn et al., 2014, Angew. Chem. Int. Ed. 53: 10975-10978; Crucianelli et al., 2014, RSC Adv. 4: 58204-58207; Das et al., 2016, Acs Macro Letters. 5: 809-813; Agarwal et al., 2016, Chem. Commun. 52: 327-330; Hyun et al., 2018, Cell Chem. Biol. 25: 1255-1267). To extend the usage of the CIM6PR/M6P system to targeted protein degradation, LYTAC was constructed by conjugating a mixture of polyglycopeptides containing 20-40 units of M6P analogues to the antibody of POI. Different from drug delivery processes, which involve the internalization of a covalent linked M6P-protein target, LYTAC allows the trafficking of a complex formed by the non-covalent interaction between the protein target and LYTAC. It was shown that LYTAC could successfully degrade both secreted and membrane proteins in the lysosome through CIM6PR (Banik et al., 2020, Nature. 584: 291-297). However, the challenge associated with the synthesis and attachment of a heterogenous mixture of polymeric glycopeptides with 20-40 units of M6P analogues to antibodies employed in the LYTAC system limited its utility in drug development. In addition, since CIM6PR is ubiquitously expressed in most cell types, the POI is delivered to all cell types non-selectively.

The present disclosure addresses the unmet need for selective means of degrading membrane and extracellular proteins. By developing new LTRs, different selectivity may be achieved for the degradation of certain membrane or extracellular protein targets depending on the expression profiles of the LTRs.

### SUMMARY

Provided herein is a bifunctional lysosomal targeting degrader, comprising a peptide ligand configured to bind to RGD-binding integrins as a shuttle molecule for lysosome degradation, and a protein binder configured to bind a pre-selected membrane or extracellular protein.

In some versions, the peptide ligand is configured to specifically bind to RGD-binding integrins as a shuttle molecule for lysosome degradation. In some versions, the protein binder is configured to specifically bind a pre-selected membrane or extracellular protein.

In one version, the peptide ligand is a cyclic peptide. An exemplary peptide ligand disclosed herein is cyclo(Arg-Gly-Asp-D-Phe-Lys) (SEQ ID NO:1).

In some embodiments, the protein binder of the bifunctional degrader binds a membrane protein. The membrane protein may be a membrane receptor. For example, the membrane receptor may be (by way of example and not limitation) epidermal growth factor receptor (EGFR).

In some embodiments, the protein binder of the bifunctional degrader binds an extracellular protein.

The protein binder of the bifunctional degrader may be any type of moiety capable of binding to the membrane or extracellular protein to be targeted for degradation via the endosomal/lysosomal pathway. For example, the protein binder is a polypeptide, a ligand, an aptamer, a nanoparticle, or a small molecule.

In some embodiments, the protein binder of the bifunctional degrader is a polypeptide. For example, the protein binder can be an antibody, either a whole antibody or a fragment of an antibody wherein the fragment retains protein-binding activity. In a specific version of the bifunctional lysosomal targeting degrader, the antibody is configured to bind an EGFR protein. In one embodiment, the antibody is Cetuximab.

The bifunctional lysosomal targeting degrader may further comprise one or more linkers to facilitate attaching the peptide ligand to the protein binder. In some embodiments, the linker is poly(ethylene glycol).

Also provided herein is a pharmaceutical composition that comprises any of the bifunctional lysosomal targeting degraders of the present disclosure. The pharmaceutical composition may further comprise a pharmaceutically acceptable carrier.

Also provided herein is a method of degrading a membrane or extracellular protein, comprising contacting the membrane or extracellular protein with any of the bifunctional lysosomal targeting degraders of the present disclosure, wherein the bifunctional lysosomal targeting degrader shuttles the membrane or extracellular protein to the lysosome for degradation.

Also provided herein is a method that comprises administering to an individual in need thereof a therapeutically effective amount of any of the pharmaceutical compositions of the present disclosure. In some embodiments, the individual is a human. In some embodiments, the individual has a cancer.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 shows gel fluorescence analysis of neutravidin-650 (NA-650) uptake by Huh7 and MCF7 cells treated with 2 μM cRGD-biotin and 500 nM NA-650 for 6 h, compared to treatment without the cRGD-biotin.

FIG. 2 shows absorption of the fluorescence of NA-650 in B16F10, Huh7, and MCF7 cells treated with 2 μM cRGD-biotin (blue bars) or triGalNAc (GN)-biotin (black bars), and 500 nM NA-650 for 24 h. Treatment without any degraders (yellow bars) are negative controls.

FIG. 3 shows co-localization of NA-650 with lysosome tracker in cells treated with 2 μM cRGD-biotin and 500 nM NA-650 for 4 h (left panel) and 24 h (right panel).

FIG. 4 shows a western blot of EGFR in HepG2 cells treated with 10 or 100 nM cRGD attached to Cetuximab (Ctx) with different length of PEG linkers (cRGD-PEG3-Ctx and cRGD-PEG12-Ctx) for 24 h. The treatment was compared to folate-based degrader Ctx-FA and transferrin-based degraders TF-P7-PEG12-Ctx, TF-P9-PEG12-Ctx, and TF-P12-PEG12-Ctx. Actin is used as loading control. “−” represents negative controls.

FIG. 5 shows a western blot of EGFR in MCF7 cells treated with 10 or 100 nM cRGD attached to Cetuximab (Ctx) with different length of PEG linkers (cRGD-PEG3-Ctx and cRGD-PEG12-Ctx) for 24 h. The treatment was compared to a folate-based degrader Ctx-FA and transferrin-based degraders TF-P7-PEG12-Ctx, TF-P9-PEG12-Ctx, and TF-P12-PEG12-Ctx. Actin is used as loading control. “−” represents negative controls.

FIG. 6 shows a western blot of EGFR in MCF7 cells treated with 10 nM cRGD attached to Cetuximab (Ctx) with different length of PEG linkers (cRGD-PEG3-Ctx and cRGD-PEG12-Ctx) for 24 h. The treatment was compared to a folate-based degrader Ctx-FA and transferrin-based degraders TF-P7-PEG12-Ctx, TF-P9-PEG12-Ctx, and TF-P12-PEG12-Ctx. Actin is used as loading control. “−” represents negative controls.

FIG. 7 shows a western blot of EGFR in Hela cells treated with 10 nM cRGD attached to Cetuximab (Ctx) with different length of PEG linkers (cRGD-PEG3-Ctx and cRGD-PEG12-Ctx) for 24 h. The treatment was compared to a folate-based degrader Ctx-FA and transferrin-based degraders TF-P7-PEG12-Ctx, TF-P9-PEG12-Ctx, and TF-P12-PEG12-Ctx. Actin is used as loading control. “−” represents negative controls.

FIG. 8 shows MCF dose response for Ctx-cRGD at 0 (control), 0.01, 0.1, 1, 10, and 100 nM.

FIG. 9 shows MCF time course response for Ctx-cRGD over 48 hours.

FIG. 10 is a fluorescence micrograph showing co-localization of EGFR and lysosome marker for Ctx and Ctx-cRGD.

FIG. 11 shows individual and merged fluorescence micrographs showing co-localization of EGFR, LAMP1, DAPI, and the merged image of all three.

FIG. 12 is a gel depicting degradation of PDL1 with Atz-PEG3-cRGD and Atz-PEG12-cRGD.

FIG. 13 is gel depicting competition of Ab-cRGD-induced anti-biotin-647 uptake by 6 μM of cRGD-azide.

FIG. 14 is a gel depicting inhibition of lysosomal degradation of anti-biotin-647 by 50 nM of Bafilomycin A1 (BAF1).

FIG. 15 is a gel depicting inhibition of EGFR degradation by increasing concentrations of cRGD-azide.

FIG. 16 is a gel depicting inhibition of EGFR degradation by lysosomal degradation inhibitors Bafilomycin A1 (BAF1, 50 nM) and Chloroquine (CQ, 10 uM).

FIG. 17 shows gels (A, C, E) and gel quantitations (B, D, F) depicting Ctx-cRGD has higher degradation efficacy on cancer cells (Hela (A, B) and HepG2 (C, D)) over normal cells (HACAT (E, F)).

### DETAILED DESCRIPTION

Provided herein are bifunctional lysosome targeting degraders that comprise: (a) a peptide ligand that binds to RGD-binding integrin as a shuttle molecule for lysosome degradation, and (b) a protein binder that binds a membrane or extracellular protein of interest. The bifunctional degrader disclosed herein finds use, e.g., for selectively targeted degradation of membrane and extracellular proteins via the endosomal/lysosomal pathway. In one aspect, the bifunctional degraders induce degradation of oncogenic proteins specifically in cancer cells through the RGD-binding integrin. Also provided herein are compositions comprising the bifunctional degraders, as well as methods of using the bifunctional degraders to inhibit disease states, including cancers.

It is to be understood that the bifunctional degraders, compositions, and methods disclosed herein are not limited to particular embodiments described, as such may, of course, vary. The bifunctional degraders, compositions, and methods disclosed herein my comprise, consist of, or consist essentially of the various elements or steps disclosed herein. It is also to be understood that the terminology used herein is for the purpose of describing particular embodiments only and is not intended to be limiting.

It is noted that, as used herein and in the appended claims, the singular forms “a”, “an”, and “the” include plural referents unless the context clearly dictates otherwise. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All patents and publications referenced or mentioned herein are indicative of the levels of skill of those skilled in the art to which the disclosure pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

It is appreciated that certain features of the bifunctional degraders, compositions, and methods, which are, for clarity, described in the context of separate embodiments, may also be provided in combination in a single embodiment. Conversely, various features of the bifunctional degraders, compositions, and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present bifunctional degraders, compositions, and methods and are disclosed herein just as if each and every such sub-combination was individually and explicitly disclosed herein.

### Bifunctional Lysosome Targeting Degraders

Provided herein are bifunctional lysosome targeting degraders that comprise a peptide ligand that binds to RGD-binding integrin as a shuttle molecule for lysosome degradation, and a protein binder that binds a membrane or extracellular protein of interest.

Integrins are heterodimeric transmembrane glycoproteins that include one α- and one β-subunit. The α- and β-subunits are bound in a noncovalent complex with the ligand-binding site at the interface. Integrins act as adhesion receptors, with the ability to signal in both directions across the plasma membrane. These events are called “inside-out” signaling and “outside-in” signaling, resulting either from binding to extracellular ligands or from interacting with the cytoskeleton via the integrin intracellular domains. Integrins can therefore enable human cells to respond to changes in the extracellular environment (via outside-in signaling) and can influence the extracellular environment itself (via inside-out signaling). See Hynes, 2002, Cell 110: 673-687; Zhu et al., 2007, Blood 110: 2475-2483; and Slack et al., 2022, Nat. Rev. Drug Discov. 21: 60-78. Among the 24 human integrin subtypes known to date, eight integrin dimers, i.e., αvβ1, αvβ3, αvβ5, αvβ6, αvβ8, α5β1, α8β1, and αIIbβ3, recognize the tripeptide Arg-Gly-Asp (RGD) motif within extracellular matrix proteins.

Peptide ligands that can bind to integrins have been extensively investigated for the delivery of anti-cancer drugs. See Nieberler et al., 2017, Cancers (Basel) 9: 116; Hatley et al., 2018, Chem. Int. Ed 57: 3298-3321; Sani et al., 2021, Chembiochem 22: 1151-1160; and Ludwig et al., 2021, Cancers (Basel) 13: 1711. In the present disclosure, it is shown that peptide ligands that bind to RGD-binding integrins can be used for the development of lysosome targeting degraders. As disclosed herein, the peptide ligand is attached to a binder of a membrane or extracellular protein of interest, creating a lysosome targeting degrader. Once inside the lysosome, the target protein is released from the molecule and degraded. The shuttling molecule is released back outside of the cell where it can bind another target protein.

Any peptide ligands that bind RGD-binding integrins are contemplated to be useful herein, including linear and cyclic peptides.

In one version of the disclosure, the peptide ligand is a cyclic peptide that binds RGD-binding integrins. Cyclization of a peptide can increase the stability and reduce the conformational space to improve the biological potency of the molecule. Non-limiting examples of cyclic peptide that binds RGD-binding integrins include c(RGDfK) (SEQ ID NO:1), c(RGDfV) (SEQ ID NO:2), c(RGDfE) (SEQ ID NO:3), c(RGDyK) (SEQ ID NO:4), c(RGDfC) (SEQ ID NO:5), c(phgisoDGRk) (SEQ ID NO:6), c(RGDf(NMe)V) (SEQ ID NO:7), and c(FRGDLAFp(NMe)K)(SEQ ID NO:8). See Kapp et al., 2017, Sci. Rep. 7: 39805.

In one embodiment, the cyclic peptide is c(RGDfK)=cyclo(Arg-Gly-Asp-D-Phe-Lys) (SEQ ID NO:1). The key residues are Arg-Gly-Asp or RGD, while Lys is part of the linker. This cyclic peptide has a strong affinity to αvβ3 isoform (2 nM), moderate binding to αvβ5, αvβ6, α5β1 (50-350 nM), and weak binding to αvβ8 and αIIbβ3 (>5,000 nM) (Kapp et al., 2017, Sci. Rep. 7: 39805).

As disclosed herein, the bifunctional lysosome targeting degraders include a protein binder that binds a membrane or extracellular protein of interest.

In some embodiments, the protein binder binds a membrane protein.

In certain embodiments, the membrane protein is a membrane receptor. Membrane receptors of interest include, but are not limited to, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1).

In a specific version, the membrane receptor is EGFR, which is known to be frequently mutated or overexpressed in different types of human cancers (Yarden and Pines, 2012, Nat Rev Cancer. 12: 553-563; Sigismund et al., 2018, Mol. Oncol. 12: 3-20).

The membrane protein may be an immune inhibitory receptor. As used herein, an “immune inhibitory receptor” is a receptor present on an immune cell that negatively regulates an immune response. Examples of inhibitory immune receptors include immune inhibitory receptors of the Ig superfamily, including but not limited to: CD200R, CD300a (IRp60; mouse MAIR-I), CD300f (IREM-1), CEACAM1 (CD66a), FcyRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-α (FDF03), SIRL-1, and SIRP-α. Further examples of immune inhibitory receptors include sialic acid-binding Ig-like lectin (Siglec) receptors, e.g., Siglec 7, Siglec9, and/or the like. Additional examples of immune inhibitory receptors include C-type lectins, including but not limited to: CLEC4A (DCIR), Ly49Q and MICL. Details regarding immune inhibitory receptors may be found, e.g., in Steevels et al., 2011, Eur. J. Immunol. 4:575-587.

The membrane protein may optionally be a ligand of an immune inhibitory receptor, one example of which is CD47, which binds to SIRP-α to prevent phagocytosis and known to be overexpressed in cancer cells (Eladl et al., 2020, J. Hematol. Oncol. 13: 96).

The membrane protein may also be an immune checkpoint molecule including immune checkpoint proteins and ligands. Non-limiting examples of immune checkpoint molecules include PD-1, PD-L1, CTLA4, TIM3, LAG3, TIGIT, and a member of the B7 family. In one embodiment, the membrane protein is PD-L1 (Programmed Cell Death Ligand 1), which binds PD-1 (programmed cell death-1) to inhibit apoptosis and known to be overexpressed in cancer cells (Yi et al., 2021, J. Hematol. Oncol. 14: 10).

In some embodiments, the protein binder binds an extracellular protein.

The extracellular protein may be a ligand for a membrane receptor. Membrane receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor β (TGF-β), including any particular subtypes of such cytokines), hormones, and the like.

Alternatively, the extracellular protein may be an antibody e.g., an antibody that binds a membrane protein or a different extracellular protein. The antibody may be an auto-antibody. By “auto-antibody” is meant an antibody produced by the immune system that is directed against one or more of the individual's own proteins. Cancer cells can induce an immunological response resulting in the production of tumor-associated auto-antibodies. Non-limiting examples of auto-antibodies include rheumatoid factor (RF), antinuclear antibody (ANA), antineutrophil cytoplasmic antibodies (ANCA), anti-double stranded DNA (anti-dsDNA), anticentromere antibodies (ACA), anticyclic citrullinated peptide antibodies (anti-CCP), extractable nuclear antigen antibodies (ENA), anticardiolipin antibodies, beta-2 glycoprotein 1 antibodies, antiphospholipid antibodies (APA), lupus anticoagulants (LA), anti-tissue transglutaminase (anti-tTG), anti-gliadin antibodies (AGA), intrinsic factor antibodies, parietal cell antibodies, thyroid antibodies, smooth muscle antibodies (SMA), antimitochondrial antibodies (AMA), anti-glomerular basement membrane (GBM), acetylcholine receptor (AChR) antibodies, etc.

The extracellular protein may be a secreted protein, including, but not limited to, secreted growth factors, extracellular matrix-degrading proteinases, cell motility factors and immunoregulatory cytokines or other bioactive molecules.

The extracellular protein may also be a mutated protein.

When the protein binder of the bifunctional degrader binds a membrane or extracellular protein, the membrane or extracellular protein may be present on a cancer cell or produced by a cancer cell. By “cancer cell” is meant a cell exhibiting a neoplastic cellular phenotype, which may be characterized by one or more of, for example, abnormal cell growth, abnormal cellular proliferation, loss of density-dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell,” “neoplastic cell,” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. In some embodiments, the membrane protein present on the cancer cell is a tumor-associated antigen or a tumor-specific antigen.

The protein binder of the bifunctional degrader may be any type of moiety capable of binding to the membrane or extracellular protein to be targeted for degradation via the endosomal/lysosomal pathway. In certain aspects, the protein binder is selected from a polypeptide, a ligand (e.g., a ligand for a membrane receptor, where the membrane receptor is targeted for degradation), an aptamer, a nanoparticle, and a small molecule.

The protein binder may be a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less.

The protein binder may be a polypeptide, such as an antibody. The terms “antibody” and “immunoglobulin” include antibodies or immunoglobulins of any isotype (e.g., IgG (e.g., lgG1, lgG2, lgG3 or lgG4), IgE, IgD, IgA, IgM, etc.); whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the membrane or extracellular protein, including, but not limited to, Fv, single chain Fv (scFv), Fab, F(ab′)₂, Fab′, (scFv′)₂, diabodies, and nanobodies; chimeric antibodies; monoclonal antibodies; fully human antibodies; humanized antibodies (e.g., humanized whole antibodies, humanized antibody fragments, etc.); and fusion proteins including an antigen-binding portion of an antibody and a non-antibody protein or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

In certain versions, the antibody is configured to bind a cancer antigen specifically.

The antibody may also be configured to bind to an intact complement or a fragment thereof. In certain embodiments, the antibody binds to one or more immunodominant epitope(s) within intact complement or a fragment thereof.

Alternatively, the antibody may bind to a membrane receptor or a membrane receptor ligand. Or the antibody may bind to an epidermal growth factor (EGF) protein, e.g., a human EGF, or one or more immunodominant epitope(s) within an EGF protein.

In certain embodiments, the antibody binds to an EGFR protein. In certain embodiments, the antibody binds to one or more immunodominant epitope(s) within an EGFR protein. In a certain embodiment, the antibody comprises the CDRs present in Cetuximab (Ctx). In another certain embodiment, the antibody comprises the variable light chain and variable heavy chain present in Cetuximab. In a particular embodiment, the antibody is Cetuximab.

In certain embodiments, the antibody binds to an immune inhibitory receptor. In certain embodiment, the antibody binds to one or more immunodominant epitope(s) within an immune inhibitory receptor.

In certain embodiments, the antibody binds to a ligand of an immune inhibitory receptor. In certain embodiment, the antibody binds to one or more immunodominant epitope(s) within a ligand of an immune inhibitory receptor. In certain embodiments, the antibody binds to a CD47 protein. In certain embodiments, the antibody binds to one or more immunodominant epitope(s) within a CD47 protein.

In certain embodiments, the antibody binds to an immune checkpoint molecule. In certain embodiments, the antibody binds to one or more immunodominant epitope(s) within an immune checkpoint molecule. In certain embodiments, the antibody binds to a PD-L1 protein. In certain embodiments, the antibody binds to one or more immunodominant epitope(s) within PD-L1 protein. In a certain embodiment, the antibody comprises the CDRs present in Atezolizumab (Atz). In another certain embodiment, the antibody comprises the variable light chain and variable heavy chain present in Atezolizumab. In a particular embodiment, the antibody is Atezolizumab.

The bifunctional lysosome targeting degraders disclosed herein may be in any suitable format. In some embodiments, the bifunctional degrader is a conjugate. Accordingly, in certain embodiments, a bifunctional degrader disclosed herein includes the RGD-binging integrin peptide ligand conjugating to the protein binder. In some embodiments, the protein binder is a polypeptide, and the bifunctional molecule is a fusion protein comprising the RGD-binding integrin peptide ligand fused to the protein binder.

In certain embodiments, one or more linkers may be employed to facilitate attaching the RGD-binding integrin peptide ligand to the protein binder. Non-limiting examples of such linkers include ester linkers (e.g., N-hydroxysuccinimide (NHS) ester, sulfo-NHS ester or PFP ester or thioester), amide linkers, maleimide or maleimide-based linkers; valine-citrulline linkers; hydrazone linkers; N-succinimidyl-4-(2-pyridyldithio)butyrate (SPDB) linkers; Succinimidyl-4-(A/-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof. In one embodiment, the linker is PEG. Various lengths of PEG may be used as linkers, such as PEG3, PEG 12, etc.

In certain aspects, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzoyloxy (Val-Ala-PAB) linker, and the like. Chemically-labile linkers, enzyme-labile, and non-cleavable linkers are known and described in detail, e.g., in Ducry and Stump, 2010, Bioconjugate Chem. 21: 5-13.

In certain aspects, the bifunctional degrader enhances degradation of the membrane or extracellular protein relative to degradation of the membrane or extracellular protein in the presence of the protein binder alone. According to some embodiments, the bifunctional degrader enhances degradation of the membrane or extracellular protein relative to degradation of the membrane or extracellular protein in the presence of the RGD-binding integrin peptide ligand or the protein binder alone. By “enhances degradation” in this context means the membrane or extracellular protein is degraded in the presence of the bifunctional degrader and is not degraded in the presence of the protein binder alone, or the presence of the RGD-binding integrin peptide ligand or the protein binder alone, under the same conditions; or the membrane or extracellular protein is degraded in the presence of the bifunctional degrader to a greater extent than the membrane or extracellular protein is degraded in the presence of the protein binder alone, or the presence of the RGD-binding integrin peptide ligand or the protein binder alone, under the same conditions. When the membrane or extracellular protein is degraded in the presence of the bifunctional degrader to a greater extent than the membrane or extracellular protein is degraded in the presence of the protein binder alone, or the presence of the RGD-binding integrin peptide ligand or the protein binder alone under the same conditions, the degradation may be 1.2 fold or greater, 1.4 fold or greater, 1.6 fold or greater, 1.8 fold or greater, 2 fold or greater, 2.5 fold or greater, 3 fold or greater, 3.5 fold or greater, 4 fold or greater, 4.5 fold or greater, 5 fold or greater, 5.5 fold or greater, 6 fold or greater, 6.5 fold or greater, 7 fold or greater, 7.5 fold or greater, 8 fold or greater, 8.5 fold or greater, 9 fold or greater, 9.5 fold or greater, or 10 fold or greater in the presence of the bifunctional degrader.

### Compositions

Disclosed herein are compositions that include any of the bifunctional lysosomal targeting degraders in the present disclosure.

The compositions may optionally include a bifunctional degrader of the present disclosure present in a liquid medium. The liquid medium may be an aqueous liquid medium, such as water, a buffered solution, and the like. One or more additives such as a salt (e.g., NaCl, MgCl₂, KCL, MgSO₄), a buffering agent (a Tris buffer, N-(2-Hydroxyethyl)piperazine-N′-(2-ethanesulfonic acid) (HEPES), 2-(NMorpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a protease inhibitor, glycerol, and the like may be present in such compositions.

Also disclosed herein are pharmaceutical compositions that include any of the bifunctional lysosomal targeting degraders of the present disclosure, and a pharmaceutically acceptable carrier. The pharmaceutical compositions generally include a therapeutically effective amount of the bifunctional degrader. By “therapeutically effective amount” is meant a dosage sufficient to produce a desired result, e.g., an amount sufficient to effect beneficial or desired therapeutic (including preventative) results, such as a reduction in cellular proliferation in an individual having a cell proliferative disorder (e.g., cancer) associated with the membrane or extracellular protein to which the protein binder of the bifunctional degrader binds, etc. An effective amount may be administered in one or more administrations.

A bifunctional degrader of the present disclosure can be incorporated into a variety of formulations for therapeutic administration. More particularly, the bifunctional degrader can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the bifunctional degraders of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the bifunctional degrader can be administered alone or in appropriate association, as well as in combination, with other pharmaceutically active compounds. The following methods and excipients are merely examples and are in no way limiting.

For oral preparations, the bifunctional degrader can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bifunctional degraders can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

An aqueous formulation of the bifunctional degrader may be prepared in a pH buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Method of Use

Disclosed herein are methods of using the bifunctional lysosomal targeting degraders of the present disclosure.

Provided herein are methods of degrading a membrane or extracellular protein. Such methods include contacting the membrane or extracellular protein with any of the bifunctional lysosomal targeting degraders of the present disclosure, under conditions in which the bifunctional lysosomal targeting degrader shuttles the membrane or extracellular protein to lysosome for degradation. Such methods find use in a variety of applications. In certain aspects, the method is performed in vitro (e.g., in a tube, cell culture plate or well, or the like) and finds use, e.g., in testing and/or research applications. In other aspects, the method is performed in vivo (e.g., in an individual to whom the bifunctional degrader is administered) and finds use, e.g., in clinical/therapeutic applications.

Also provided are methods that include administering to an individual in need thereof a therapeutically effective amount of any of the bifunctional degraders or any of the pharmaceutical compositions of the present disclosure. A variety of individuals are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

An effective amount of the bifunctional degrader (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., cancer) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the bifunctional degrader or pharmaceutical composition.

The methods include administering to an individual having cancer a therapeutically effective amount of any of the bifunctional degraders or any of the pharmaceutical compositions of the present disclosure. According to such methods, the protein binder of the bifunctional degraders binds a membrane or extracellular protein that at least contributes to the individual's cancer, and where targeted degradation of the membrane or extracellular protein using the bifunctional degrader treats the individual's cancer. In certain aspects, the protein binder binds to a protein selected from a membrane receptor, a ligand for a membrane receptor, an immune inhibitory receptor, a ligand of an immune inhibitory receptor, an immune checkpoint molecule, an autoantibody, a secreted protein, and a mutated protein.

For example, the individual to be treated may have a cancer characterized by the presence of a solid tumor, a semi-solid tumor, a primary tumor, a metastatic tumor, or the like. In some embodiments, the individual has a cancer selected from breast cancer, melanoma, lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, any combinations thereof, and any sub-types thereof.

In any of the methods of using the bifunctional degraders of the present disclosure, the bifunctional degrader generally enhances degradation of the membrane or extracellular protein relative to degradation of the membrane or extracellular protein in the presence of the protein binder alone. Similarly, in any of the methods of using the bifunctional degraders of the present disclosure, according to some embodiments, the bifunctional degrader enhances degradation of the membrane or extracellular protein relative to degradation of the membrane or extracellular protein in the presence of the RGD-binding integrin peptide ligand or the protein binder alone.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g., symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

The bifunctional degrader or pharmaceutical composition may be administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the bifunctional degrader and/or the desired effect. The bifunctional degraders or pharmaceutical compositions may be administered in a single dose or in multiple doses. In some embodiments, the bifunctional degrader or pharmaceutical composition is administered intravenously. In some embodiments, the bifunctional degrader or pharmaceutical composition is administered by injection, e.g., for systemic delivery (e.g., intravenous infusion) or to a local site.

### EXAMPLES

We tested a cyclic peptide as a binder of RGD-binding integrins for the development of lysosome targeting degraders. The cyclic peptide cRGD=cyclo(Arg-Gly-Asp-D-Phe-Lys) (SEQ ID NO:1), also known as c(RGDfK) (SEQ ID NO:1) (Kapp et al., 2017, Sci. Rep. 7: 39805). The key residues are Arg-Gly-Asp or RGD, while Lys is part of the linker. This cyclic peptide has a strong affinity to αvβ3 isoform (2 nM), moderate binding to αvβ5, αvβ6, α5β1 (50-350 nM), and weak binding to αvβ8 and αIIbβ3 (>5,000 nM) (Kapp et al., 2017, Sci. Rep. 7: 39805).

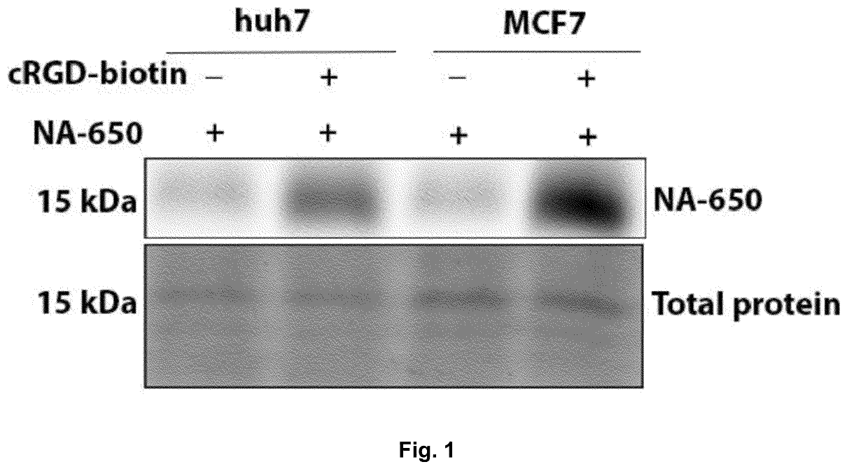

A conjugate of c(RGDfK)(SEQ ID NO:1) to biotin (cRGD-biotin, Vivitide, PCI-3697-PI-1MG) can bind to a fluorescent model target protein neutravidin-650 (NA-650). The cRGD-biotin was tested for uptake of the model target protein NA-650 by treating Huh7 and MCF7 cells with 2 μM cRGD-biotin and 500 nM NA-650 for 6 h. FIG. 1 shows that with the presence of cRGD-biotin, uptake of the NA-650 protein was observed in both Huh7 and MCF7 cells.

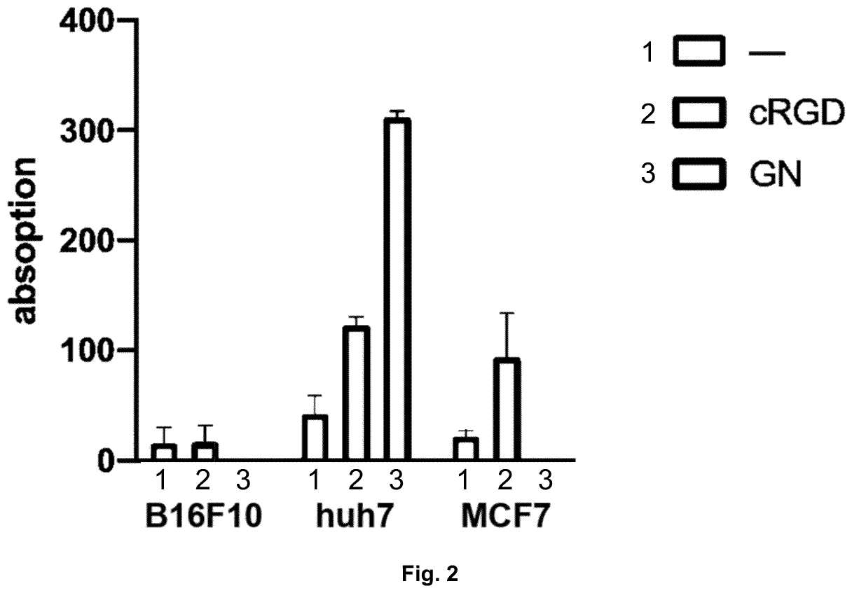

We also compared the uptake of NA-650 with triGalNAc (GN)-biotin, which is one of the most efficient lysosome targeting degraders for the uptake of secreted protein into liver cells (e.g., Huh7) (Zhou et al., 2021, ACS Cent. Sci. 7: 499). The Huh7 cells were treated with 2 μM cRGD-biotin or GN-biotin and 500 nM NA-650 for 24 h, and the results were compared to the treatment of non-liver cells B16F10 and MCF7. As shown in FIG. 2, with the presence of GN-biotin, only Huh7 cells uptake NA-650. With the presence of cRGD-biotin, all three types of cells uptake NA-650.

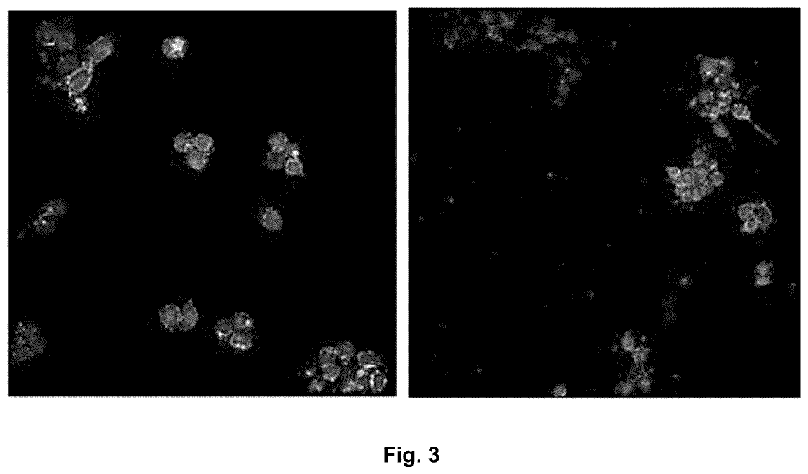

We also observed the co-localization of the NA-650 protein with lysosome tracker as shown in the images of FIG. 3. The results demonstrate that the cyclic peptide-based lysosome targeting degraders can promote the uptake of soluble model target protein into the lysosome.

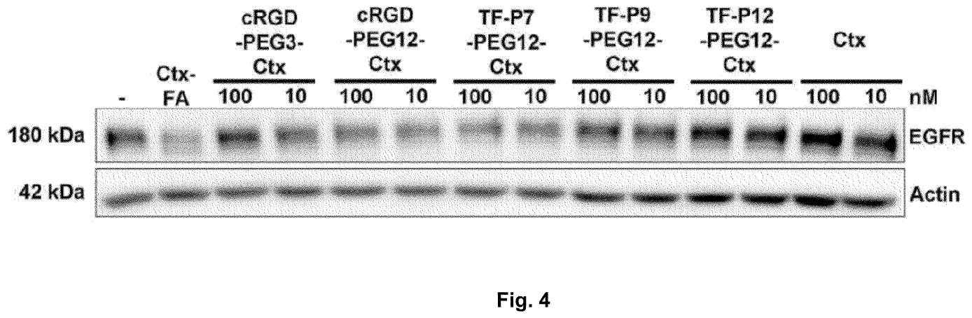

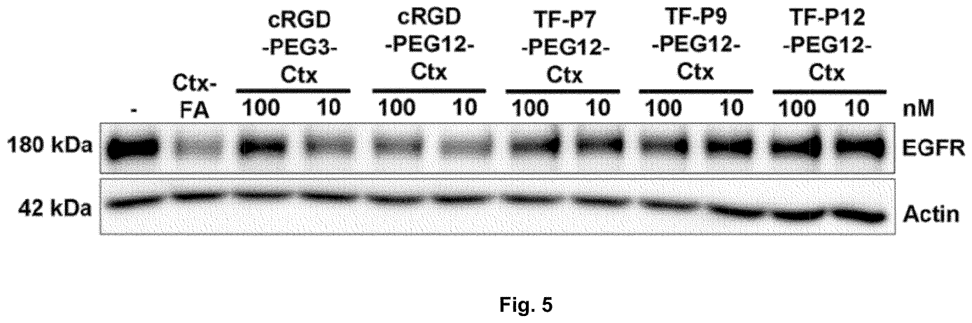

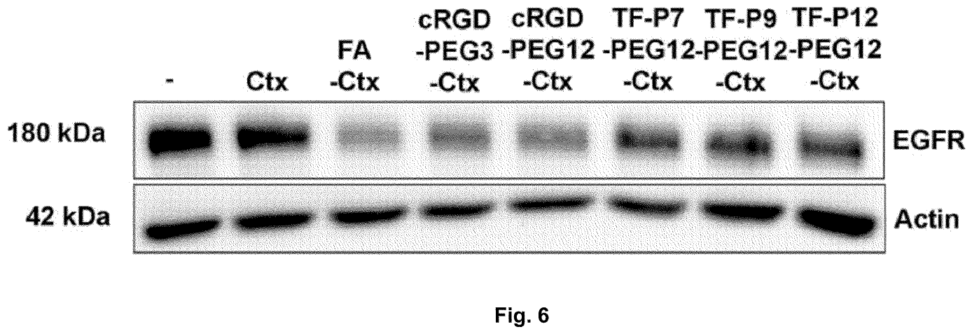

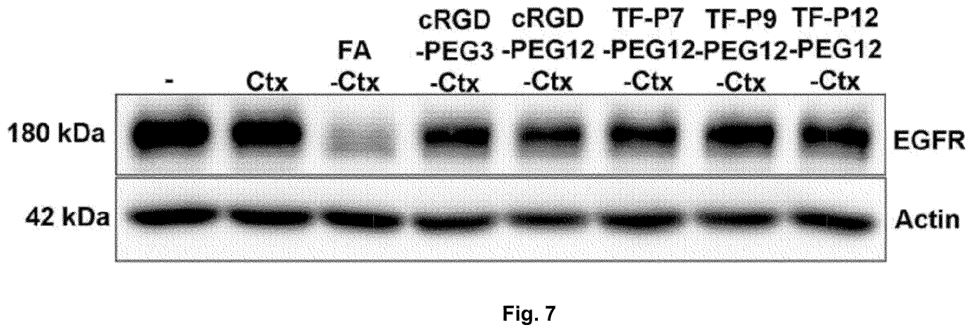

We also attached the cRGD peptide to Cetuximab (Ctx), an antibody that can bind to a membrane target protein EGFR. The lysosome targeting degraders were prepared and tested for the degradation of EGFR. We used two linkers between the cyclic peptide binder for the receptor and the antibody, the binder of EGFR. The linkers were polyethylene glycol 3 (PEG3; H—(O—CH₂—CH₂)₃—OH) and polyethylene glycol 12 (PEG12; H—(O—CH₂—CH₂)₁₂—OH). A construct of Cetuximab conjugated to folate via a PEG3 linker (Ctx-FA) was used as a positive control. For the preparation of the cRGD-Cetuximab conjugates (cRGD-PEG3-Ctx, cRGD-PEG12-Ctx), Cetuximab was reacted at a concentration of 1.8 mg/ml in 200 μL PBS with DBCO-PEG3-NHS or DBCO-PEG12-NHS ester at 1:25 molar ratio overnight at room temperature on a rotator. The mixture was then purified with 500 μL of PBS for 5 times using 10 kDa Amicon Centrifugal Filter, followed by measuring the concentration of DBCO-labeled antibody by BCA assay and reacting with cRGD-N3 overnight at room temperature on a rotator. The resulting antibody conjugate was then purified with 500 μL of PBS for 5 times using 10 kDa Amicon Centrifugal Filter. The purified antibody conjugate was then conjugated to cRGD via the NHS moiety. For the preparation of Ctx-FA, Cetuximab was reacted at the concentration of 1.8 mg/ml in 200 μL PBS with DBCO-PEG3-NHS ester at 1:25 molar ratio overnight at room temperature on a rotator. The mixture was then purified with 500 μL of PBS for 5 times using 10 kDa Amicon Centrifugal Filter, followed by measuring the concentration of DBCO-labeled antibody by BCA assay and reacting with Folate-N3 overnight at room temperature on a rotator. The resulting antibody conjugate was then purified with 500 μL of PBS for 5 times using 10 kDa Amicon Centrifugal Filter. The degradation results (FIGS. 4-7) show a significant decrease in EGFR protein levels in cells treated with the cyclic peptide cRGD labeled antibody, suggesting that EGFR has been degraded in the cells. Degrader with a longer PEG linker (PEG12) has better degradation activity for EGFR in the tested cancer cells. Less degradation effect was observed at 100 nM than 10 nM concentration, which is likely due to hook effect (Douglass et al., 2013, J. Am. Chem. Soc. 135: 6092).

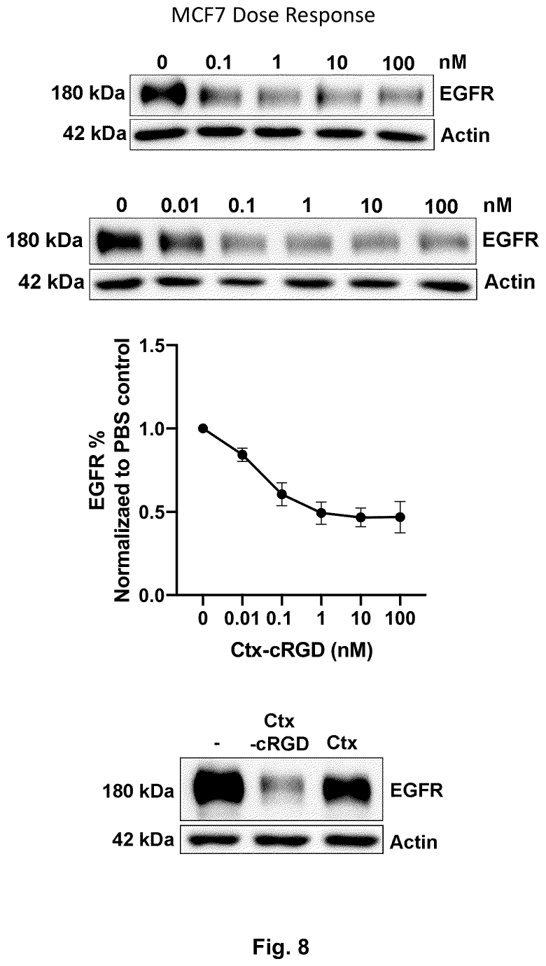

MCF dose response experiments with the DBCO-PEG12-NHS construct (also referred to herein as Ctx-cRGD) at 0 (control), 0.01, 0.1, 1, 10, and 100 nM. MCF7 cells were seeded at 70% confluence in a 24-well plate and incubated in 350 μL complete culture media overnight. Then, cells were treated with Ctx-PEG12-cRGD in 50 μL culture media at various concentrations as indicated in the figure for 24 h before collection for western blot analysis. Results are shown in FIG. 8.

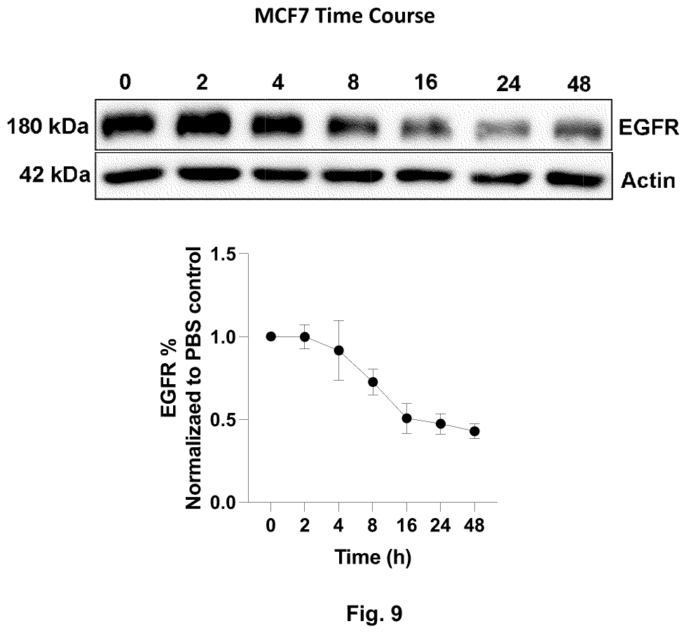

MCF time course response for Ctx-cRGD was determined over 48 hours. MCF7 cells were seeded at 70% confluence in a 24-well plate and incubated in 350 μL complete culture media overnight. Then, cells were treated with Ctx-PEG12-cRGD in 50 μL culture media at 10 nM for different periods as indicated in the figure before collection for western blot analysis. Results are shown in FIG. 9.

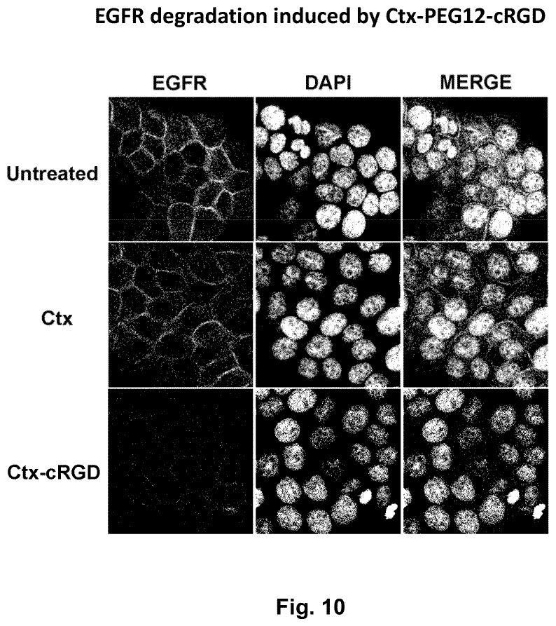

FIG. 10 is a fluorescence micrograph showing co-localization of EGFR and lysosome marker for Ctx and Ctx-cRGD. MCF7 cells at the density of 20,000 cells/well was distributed onto 8-well chamber slides in 200 μL of complete culture medium. Cells were treated with 10 nM Ctx-PEG12-cRGD for 24 h at 37° C., followed by 3 washes with PBS. Cells were then fixed with 4% paraformaldehyde for 15 min followed by permeabilization with 0.5% Triton-100 for 5 min and blocking with 5% BSA for 1 h at RT. Cells were then incubated with anti-EGFR antibody in 1% BSA overnight at 4° C. The next day, cells were incubated with anti-rabbit-594 secondary antibody for 1 h at RT. Cells was washed 3 times with PBS after each antibody incubation. Then the cells were mounted with slowfade-antifade mounting medium containing DAPI. Images were acquired by Leica SP8 3×STED super-resolution microscope at 60× magnification with a 10× eyepiece and analyzed by ImageJ.

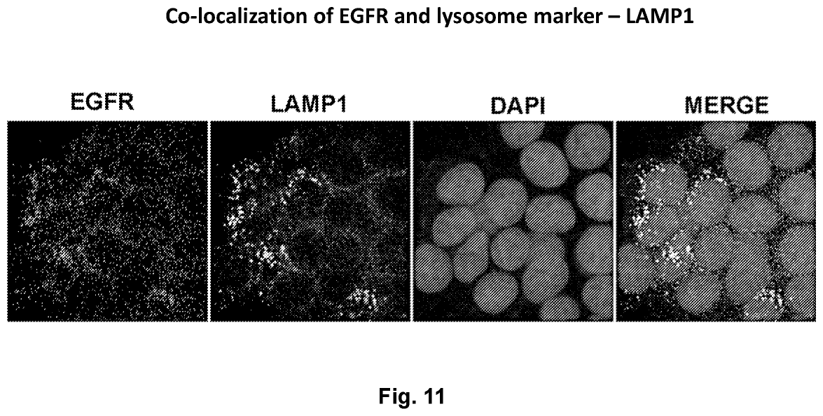

FIG. 11 shows individual and merged fluorescence micrographs showing co-localization of EGFR, LAMP1, DAPI, and the merged image of all three. MCF7 cells at the density of 20,000 cells/well was distributed onto 8-well chamber slides in 200 μL of complete culture medium. Cells were treated with 10 nM Ctx-cRGD for 24 h at 37° C., followed by 3 washes with PBS. Cells were then fixed with 4% paraformaldehyde for 15 min followed by permeabilization with 0.5% Triton-100 for 5 min and blocking with 5% BSA for 1 h at RT. Cells were then incubated with anti-EGFR antibody and anti-LAMP1 antibody in 1% BSA overnight at 4° C. The next day, cells were incubated with anti-mouse-488 and anti-rabbit-594 secondary antibody for 1 h at RT. Cells was washed 3 times with PBS after each antibody incubation. Then the cells were mounted with slowfade-antifade mounting medium containing DAPI. Images were acquired by Leica SP8 3×STED super-resolution microscope at 60× magnification with a 10× eyepiece and analyzed by ImageJ.

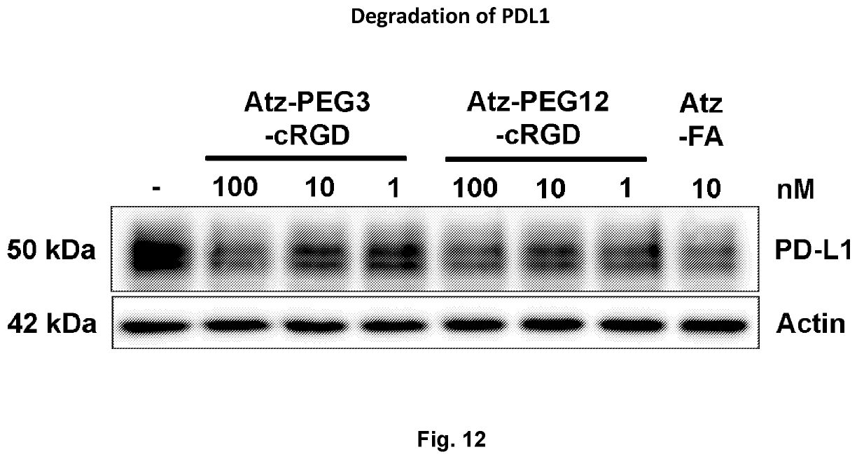

FIG. 12 shows a gel depicting degradation of PDL1 with Atz-PEG3-cRGD and Atz-PEG12-cRGD. The Atz-PEG3-cRGD and Atz-PEG12-cRGD constructs are Atezolizumab (Atz) attached with cRGD through PEG3 or PEG12 linker. Atezolizumab with the concentration of 1.8 mg/ml in 200 μL PBS was reacted with DBCO-PEG3-NHS or DBCO-PEG12-NHS ester at 1:25 molar ratio overnight at room temperature on a rotator. The mixture was then purified with 500 μL of PBS for 5 times using 10 kDa Amicon Centrifugal Filter, followed by measuring the concentration of DBCO-labeled antibody by BCA assay and reacting with cRGD-N3 overnight at room temperature on a rotator. The resulting antibody conjugate was then purified with 500 μL of PBS for 5 times using 10 kDa Amicon Centrifugal Filter. MCF7 cells were seeded at 70% confluence in a 24-well plate and incubated in 350 μL complete culture media overnight. Then, cells were treated with Atz-PEG3-cRGD or Atz-PEG12-cRGD in 50 μL culture media at various concentrations as indicated in the figure for 24 h before collection for western blot analysis. Atz-PEG3-FA treated at 10 nM was used as positive control.

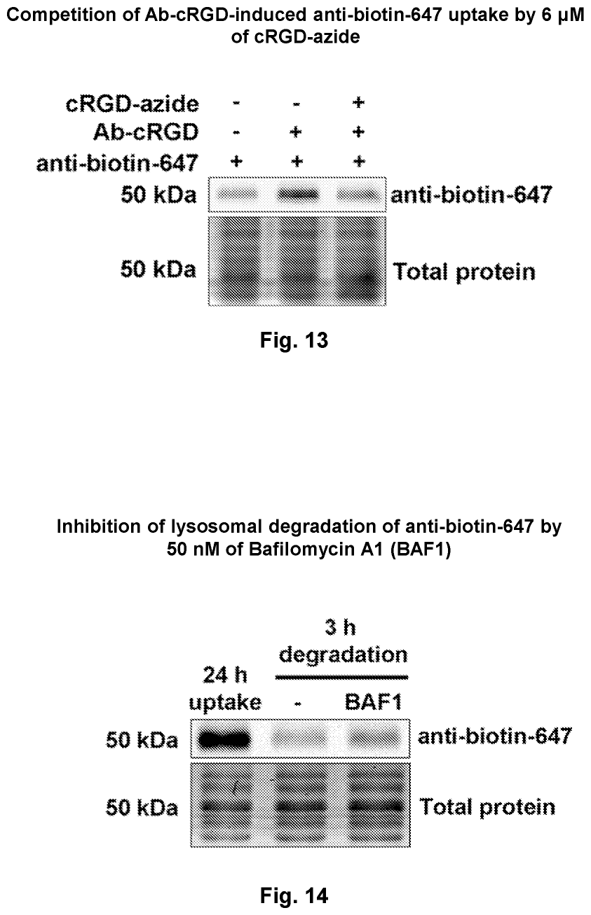

FIG. 13 is gel depicting competition of Ab-cRGD-induced anti-biotin-647 antibody (Jackson ImmunoResearch, 200-602-211) uptake by 6 μM of cRGD-azide (Vivitide, RGD-3749-PI-5MG). Ab-cRGD is Goat anti-mouse IgG antibody (Ab) attached with cRGD through PEG12 linker. Ab with the concentration of 1.8 mg/ml in 200 μL PBS was reacted with DBCO-PEG12-NHS ester at 1:25 molar ratio overnight at room temperature on a rotator. The mixture was then purified with 500 μL of PBS for 5 times using 10 kDa Amicon Centrifugal Filter, followed by measuring the concentration of DBCO-labeled antibody by BCA assay and reacting with cRGD-N3 overnight at room temperature on a rotator. The resulting antibody conjugate was then purified with 500 μL of PBS for 5 times using 10 kDa Amicon Centrifugal Filter. Cells were seeded in a 48-well plate at 70% confluence in 200 μL complete culture media one day before treatment. Cells were then treated sequentially with 25 μL medium containing 50 nM anti-biotin-647 and 25 μL medium containing 25 nM Ab-cRGD and incubated at 37° C. for indicated time periods, followed by washed twice with PBS before harvested for in gel fluorescence analysis. Cells were pre-incubated with free excess 6 μM cRGD-azide for 1 h at 4° C. for competition.

FIG. 14 is a gel depicting inhibition of lysosomal degradation of anti-biotin-647 by 50 nM of Bafilomycin A1 (BAF1). MCF7 cells were seeded in a 48-well plate at 70% confluence and maintained in 200 μL complete culture media. Next day, cells were incubated with 25 nM of Ab-cRGD and 50 nM of anti-biotin-647 for 3 h followed by three washes with PBS. Cells were then maintained in fresh media with or without 50 nM Bafilomycin A1 (BAF1) for another 3 h before harvested for in gel fluorescence analysis.

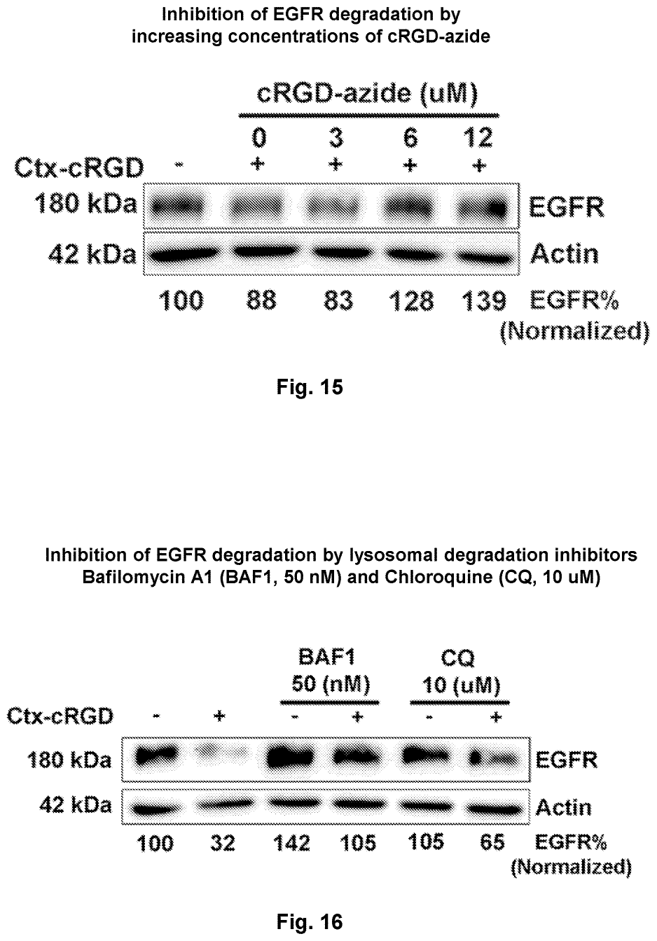

FIG. 15 is a gel depicting inhibition of EGFR degradation by increasing concentrations of cRGD-azide. MCF7 cells were seeded at 70% confluence in a 24-well plate and incubated in 350 μL complete culture media overnight. Cells were pre-treated with free excess cRGD-azide with concentrations indicated in the figure at 4° C. for 1 h before incubating with 10 nm Ctx-PEG12-cRGD for 8 h. Cells were then harvested for western blot.

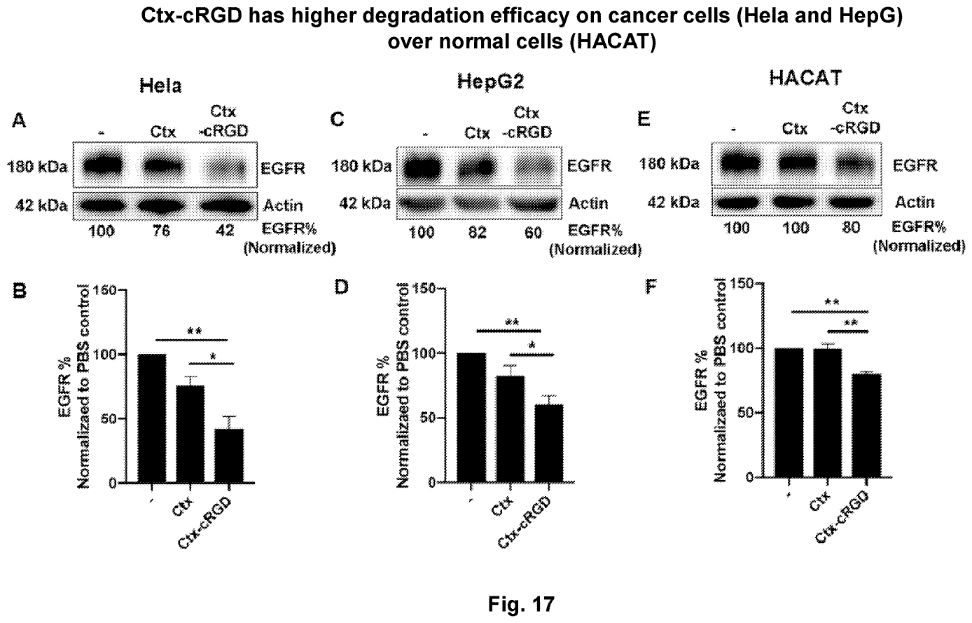

FIG. 16 is a gel depicting inhibition of EGFR degradation by lysosomal degradation inhibitors Bafilomycin A1 (BAF1, 50 nM) and Chloroquine (CQ, 10 uM). MCF7 cells were seeded at 70% confluence in a 24-well plate and incubated in 350 μL complete culture media overnight. Cells were treated with lysosomal degradation inhibitor Bafilomycin A1 (BAF1) at 50 nM and Chloroquine (CQ) at 10 uM in the presence of 10 nM Ctx-PEG12-cRGD for 6h.

FIG. 17 shows gels (A, C, E) and gel quantitations (B, D, F) depicting Ctx-cRGD has higher degradation efficacy on cancer cells (Hela (A, B) and HepG2 (C, D)) over normal cells (HACAT (E, F)). Hela, HepG2 and HACAT cells were seeded at 70% confluence in a 24-well plate and incubated in 350 μL complete culture media overnight. Cells were treated with 10 nM Ctx or Ctx-PEG12-cRGD for 24 h before collection for western blot analysis.

1. A bifunctional lysosomal targeting degrader, comprising:
a peptide ligand configured to bind to RGD-binding integrin as a shuttle molecule for lysosome degradation; and
a protein binder configured to bind a pre-selected membrane or extracellular protein.

2. The bifunctional lysosomal targeting degrader of claim 1, wherein the peptide ligand is configured to bind specifically to RGD-binding integrin as a shuttle molecule for lysosome degradation.

3. The bifunctional lysosomal targeting degrader of claim 1, wherein the protein binder is configured to bind specifically to a pre-selected membrane or extracellular protein.

4. The bifunctional lysosomal targeting degrader of claim 1, wherein the peptide ligand is a cyclic peptide.

5. The bifunctional lysosomal targeting degrader of claim 1, wherein the peptide ligand is cyclo(Arg-Gly-Asp-D-Phe-Lys) (SEQ ID NO:1).

6. The bifunctional lysosomal targeting degrader of claim 1, wherein the protein binder binds a membrane protein.

7. The bifunctional lysosomal targeting degrader of claim 6, wherein the protein binder binds a membrane receptor.

8. The bifunctional lysosomal targeting degrader of claim 7, wherein the protein binder binds epidermal growth factor receptor (EGFR).

9. The bifunctional lysosomal targeting degrader of claim 1, wherein the protein binder binds an extracellular protein.

10. The bifunctional lysosomal targeting degrader of claim 1, wherein the protein binder is a polypeptide, a ligand, an aptamer, a nanoparticle, or a small molecule.

11. The bifunctional lysosomal targeting degrader of claim 1, wherein the protein binder is a polypeptide.

12. The bifunctional lysosomal targeting degrader of claim 1, wherein the protein binder is an antibody.

13. The bifunctional lysosomal targeting degrader of claim 12, wherein the antibody is configured to bind an EGFR protein.

14. The bifunctional lysosomal targeting degrader of claim 13, wherein the antibody is Cetuximab.

15. The bifunctional lysosomal targeting degrader of claim 1, further comprising one or more linkers to facilitate attaching the peptide ligand to the protein binder.

16. The bifunctional lysosomal targeting degrader of claim 15, wherein the linker is poly(ethylene glycol).

17-18. (canceled)

19. A method of degrading a membrane or extracellular protein, comprising:
contacting the membrane or extracellular protein with the bifunctional lysosomal targeting degrader of claim 1;
wherein the bifunctional lysosomal targeting degrader shuttles the membrane or extracellular protein to lysosomes for degradation.

20. A method comprising administering to an individual in need thereof a therapeutically effective amount of the bifunctional lysosomal targeting degrader of claim 1.

21. The method of claim 20, wherein the individual is a human.

22. The method of claim 20, wherein the individual has cancer.


SEQUENCE LISTING (8 sequences)

SEQ ID NO: 1 - protein, 5 aa, synthetic construct
  SITE 4: note = D-phenylalanine
  REGION 1..5: note = Circular
RGDFK

SEQ ID NO: 2 - protein, 5 aa, synthetic construct
  SITE 4: note = D-Phenylalanine
  REGION 1..5: note = Circular
RGDFV

SEQ ID NO: 3 - protein, 5 aa, synthetic construct
  SITE 4: note = D-Phenylalanine
  REGION 1..5: note = Circular
RGDFE

SEQ ID NO: 4 - protein, 5 aa, synthetic construct
  SITE 4: note = D-Tyrosine
  REGION 1..5: note = Circular
RGDYK

SEQ ID NO: 5 - protein, 5 aa, synthetic construct
  SITE 4: note = D-Phenylalanine
  REGION 1..5: note = Circular
RGDFC

SEQ ID NO: 6 - protein, 7 aa, synthetic construct
  SITE 1: note = D-Proline
  SITE 2: note = D-Histidine
  SITE 3: note = D-Glycine
  SITE 4: note = Isoaspartate
  SITE 7: note = D-Lysine
  REGION 1..7: note = Circular
PHGDGRK

SEQ ID NO: 7 - protein, 5 aa, synthetic construct
  SITE 4: note = N-Methyl-D-Phenylalanine
RGDFV

SEQ ID NO: 8 - protein, 9 aa, synthetic construct
  SITE 8: note = N-Methyl-D-Proline
FRGDLAFPK